(19) United States
(12) Patent Application Publication
(10) Pub. No.: US 2017/0246446 A1
(43) Pub. Date: Aug. 31, 2017

(54) ELECTRICAL STIMULATION PROBE

(71) Applicant: Ho-Ching Technology Co., LTD., Taoyuan City (TW)

(72) Inventors: Wang-Su Lee, Taoyuan City (TW); Yu-Hsuan Lee, Taoyuan City (TW)

(21) Appl. No.: 15/441,772

(22) Filed: Feb. 24, 2017

Related U.S. Application Data
(60) Provisional application No. 62/300,440, filed on Feb. 26, 2016.

(51) Int. Cl. A61N 1/04; A61N 1/05

(52) U.S. Cl. CPC A61N 1/0472; A61N 1/05

(57) ABSTRACT

A hollowly cylindrical supporting body is used for an electrical stimulation probe, and the electrical stimulation probe provides an insulated outer shell. The insulated outer shell can accommodate and cover the hollowly cylindrical supporting body. Further, the hollowly cylindrical supporting body can be assembled precisely with the insulated outer shell through Fool-Proofing Design. Therefore, the insulated outer shell and the hollowly cylindrical supporting body can be combined closely and joined together to form the electrical stimulation probe of the present invention.

### CROSS-REFERENCE TO RELATED APPLICATIONS

This Non-provisional application claims priority under 35 U.S.C. §119(a) on Patent Application No. 62/300,440 filed in Feb. 26, 2016, the entire contents of which are hereby incorporated by reference.

### FIELD OF THE INVENTION

The present invention provides an electrical stimulation probe, especially related to a Fool-Proofing Designed assembled precisely electrical stimulation probe, which is used for physiotherapy and electrotherapy and/or for electric stimulation of body surface or body cavity of an organism.

### BACKGROUND OF THE INVENTION

Generally, the existed stimulation probe for physical therapy and electrotherapy is mainly manufactured by a mold combined with plastic injection molding manufacture methods. The existed method would cause convex marks on the end product, so as to make the overall appearance of the probe not smooth. Thus, the existed stimulation probes have many deficiencies and disadvantages resulted in low acceptance rates for the users' willingness to buy decreases. Even when the user purchased, the convex marks often cause the user to feel discomfort while using the existed probe on their body surface or in the body cavities.

In order to improve the appearance of the probe, the convex marks are often eliminated by the polishing step in the conventional production process. However, large amounts of dust would easily be generated during the grinding process, thereby endangers the health of manufacturing workers and easily leads to environmental pollution caused by increased aerosol particles.

The conventional manufacture process of probe products such as TWM504608 and CN 204601388 are injected hot-melt plastic materials into the inner chamber of the probe through the probe tail or into the gap appeared in the conducting wires passing into inside the probe rod from the outside. During the process of the hot-melt plastic materials flow into the interior chamber and into the gap between the solid plastic block, the hot-melt plastic would easily lead to melt the outer plastic layer of the wire, regardless of internal chamber is provided with a guide groove to guide the hot-melt plastic or not. Besides, the possibility of short-circuit would be higher in the paired wires due to too close to each other in the inner chamber. Therefore, the conventional physical therapy and electrotherapy probes of TWM504608 and CN204601388 require large amount of materials to produce, and large number of products with manual screening and eliminating inferior products is also needed, so as to increase the manufacturing time and process. The above-mentioned process results in low manufacturing efficiency and the manufacturing costs would increase significantly. Furthermore, in the manufacture process of the conventional physical therapy and electrotherapy probes in TWM504608 and CN204601388, the inner cavity of the probe is filled with a hot-melt plastic to form a solid plastic block or cladding a solid plastic block with the hot-melt plastic materials then form a solid plastic block. The weight of the probe is still too heavy, the users need to use great effort to hold the probe and is not easy to grip, which leads to inconvenient operation.

In addition, the manufacture process of the conventional physical therapy and electrotherapy probes in aforementioned TWM504608 and CN204601388 requires to pour a lot of plastic materials into the probe internal chamber. The hot-melt plastic materials (thermosetting plastics or thermoplastics) often lead to gas explosion from the probe internal chamber and induce cracks. Besides, large plastic materials would be spilled on the surface of the probe so as to reduce the product yield significantly during the manufacture process.

Furthermore, the solid plastic block is arbitrarily disposed in an existing internal cavity of the probe, the inner cavity of the probe and the solid plastic block would not be corresponded and connected with a pre-planned precision rails. Thus, the solid plastic block was restrained in the inner cavity of the probe through connecting with large amount of hot-melt plastic.

In addition, the manufacture process of the conventional physical therapy and electrotherapy probes in aforementioned TWM504608 and CN204601388 require the shell members of the probe to be joined by a large materials, such as hot melt plastics to form integral waterproof shells number of prior art techniques. However, as mentioned above, hot-melt plastics are easy to damage the wire, resulting in low yield of manufacture process. In the process of hot-melt joining, the shell components are still easy to produce micro-cracks, resulting in the existed physical therapy and electrotherapy probe with poor water resistance and high failure rate during usage. Therefore, the users often feel inconvenient and even have safety concerns.

### SUMMARY OF THE INVENTION

The problem of existing probe is the conducting wires are easily blown due to the injection molding process of hot-melt plastic materials, thus large amount of hot-melt plastic materials is needed for producing a high waterproof effect during the manufacture process. According to above problems, how to provide an electrical stimulation probe with high yield, light weighted, and with conducting wire not easily been blown by hot-melt plastic materials during the injection molding process, less hot-melt plastic materials is used to make the high waterproof, high safety and users are willing to use is an important issue. The electrical stimulation probe of the present invention greatly solved the problem of existed probe.

Accordingly, one object of the present invention is to provide an electrical stimulation probe for solving the existing problem during using and manufacture. The Fool-Proofing Design of the present invention can provide a precise assembly and match, also provide a light weighted, user friendly, safe and waterproof electrical stimulation probe.

In summary, the problem of manufacture process of existing probe is that the conducting wires are easily blown due to the injection molding process of hot-melt plastic materials, thus the large amount of hot-melt plastic materials is needed for producing a high waterproof effect. The existing manufacture process of probe is having a problem of wasting materials, long manufacture process, low yield . . . etc., Besides, the users would feel uncomfortable and inconvenient using the existed probe. To solve these problems, the electrical stimulation probe of the present invention could assemble all the components precisely by the Fool-Proofing Designed guide rails, so as to provide an electrical stimulation probe with high safety, light-weighted, easily operated, and waterproof function.

In view of above mentioned reasons, the present invention provides an electrical stimulation probe which comprises a hollowly cylindrical supporting body, and an insulated outer shell, conducting members, a handheld portion, conducting wires, and O rings.

### BRIEF DESCRIPTION OF THE DRAWINGS

FIG. 1 illustrates the decomposition diagram of one embodiment of the present invention.

FIG. 2 illustrates the three-dimensional explosion diagram of one embodiment of the present invention.

FIG. 3 illustrates the partial decomposition diagram of one embodiment of the present invention.

FIG. 4 illustrates the lateral view of three-dimensional diagram of the hollow cylindrical supporting body in another embodiment of the present invention.

FIG. 5 illustrates another lateral view of three-dimensional diagram of the hollow cylindrical supporting body in another embodiment of the present invention.

FIG. 6 illustrates the three-dimensional diagram of another embodiment of the present invention.

FIG. 7 illustrates the three-dimensional partial decomposition diagram of another embodiment of the present invention.

FIG. 8 illustrates the plan diagram of another embodiment of the present invention.

### DETAILED DESCRIPTION OF THE INVENTION

The detailed description provided below in connection with the appended drawings is intended as a description of the present examples and is not intended to represent the only forms in which the present example may be utilized or constructed. The description sets forth the functions of the example and the sequence of steps for constructing and operating the example. However, the same or equivalent functions and sequences may be accomplished by different examples.

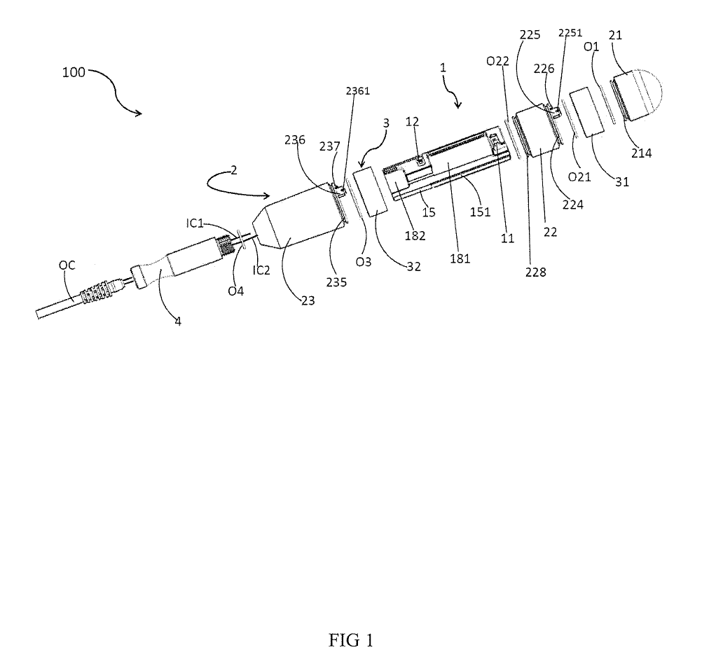

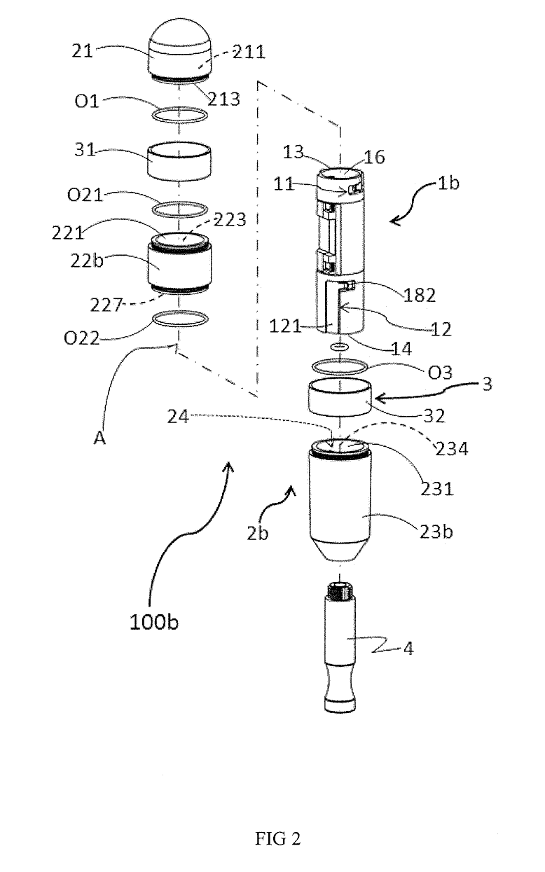

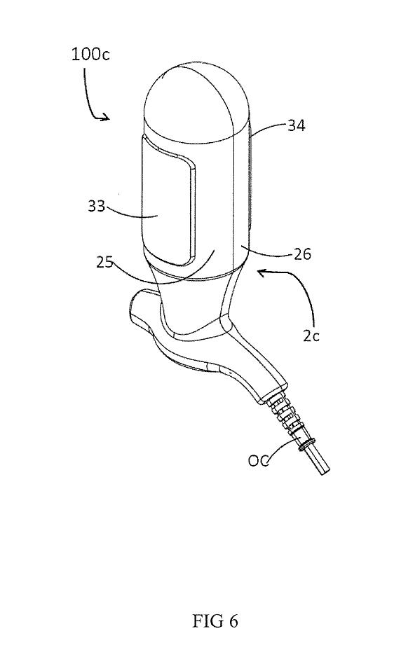

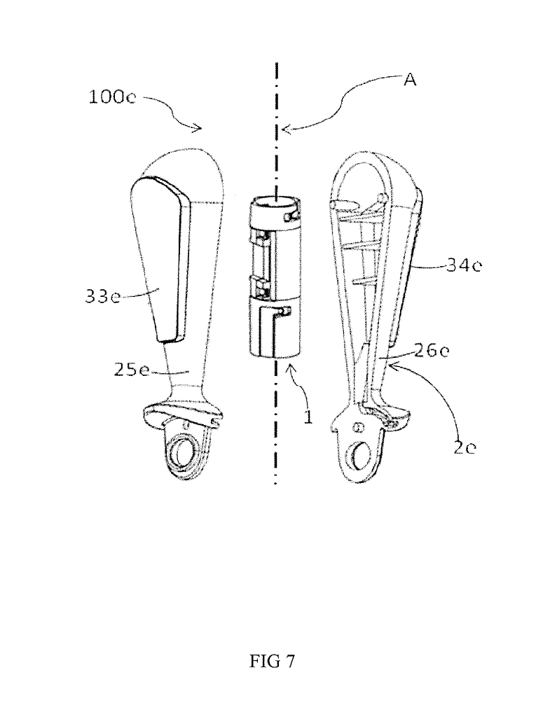

Please refer to FIG. 1, one embodiment of the present invention, the electrical stimulation probe 100 includes a hollow cylindrical supporting body 1 and an insulated outer shell 2. The insulated outer shell 2 is covering, encompassing and connected to the hollow cylindrical supporting body 1. The outer shell of the insulated outer shell 2 is having a plurality conducting member 3. The electrical stimulation probe 100 of the present invention is electrically connected with outside electrical power through outer conducting wire OC, and an outer conducting wire OC is connected with a handheld portion 4. The plurality of conducting members 3 is the combination of at least two conducting rings, at least two conducting members, or at least one conducting ring and at least one conducting member. The conducting member 3, for example, but not limited to, two conducting rings, three conducting rings, two conducting sheets, three conducting sheets, one conducting ring and one conducting sheet, two conducting rings and a conducting sheet, a conducting ring and two conducting sheets etc. That is, in one embodiment of the present invention, the plurality of the conducting members is an upper conducting ring 31 and a lower conducting ring 32 (as shown in FIG. 1 and FIG. 2). In another embodiment of the present invention, the plurality of the conducting members can be a first conducting sheet 33, 33e and a second conducting sheet 34, 34e (as shown in FIG. 6 and FIG. 7).

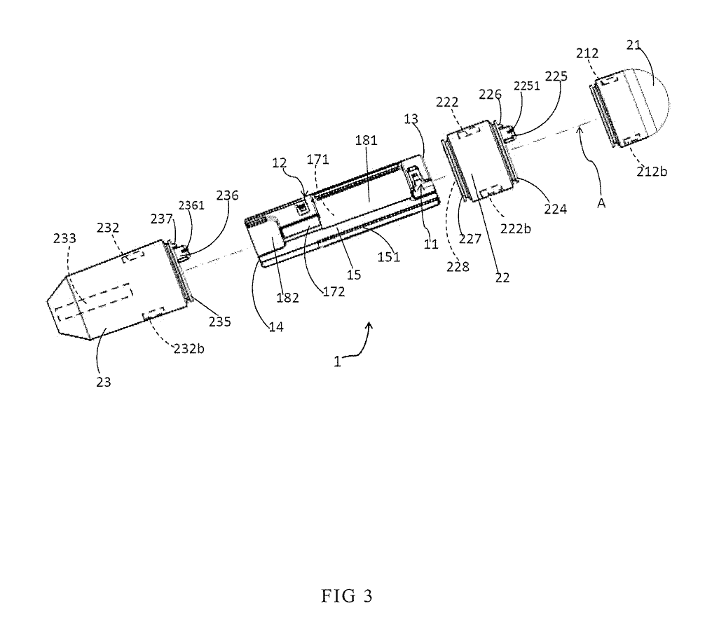

Furthermore, please refer to FIG. 2 and FIG. 3, an inward part of the insulated outer shell 2 has a hollow portion 24. The hollow cylindrical supporting body 1 slides along the hallow portion 24, and the hollow cylindrical supporting body 1 is coated and supported by the insulated outer shell 2. The insulated outer shell 2 is sequentially assembled in the axial direction or parallel to the axial direction by a plurality of shell members. In one of the embodiment of the present invention, the insulated outer shell 2 is assembled sequentially along an axis direction A (as shown in FIG. 1 and FIG. 2). In another embodiment, the insulated outer shell 2 is assembled in a symmetrical mirror, or asymmetric manner by a plurality of shell members corresponding with paralleling to the axis direction A (as shown in FIG. 6 and FIG. 7)

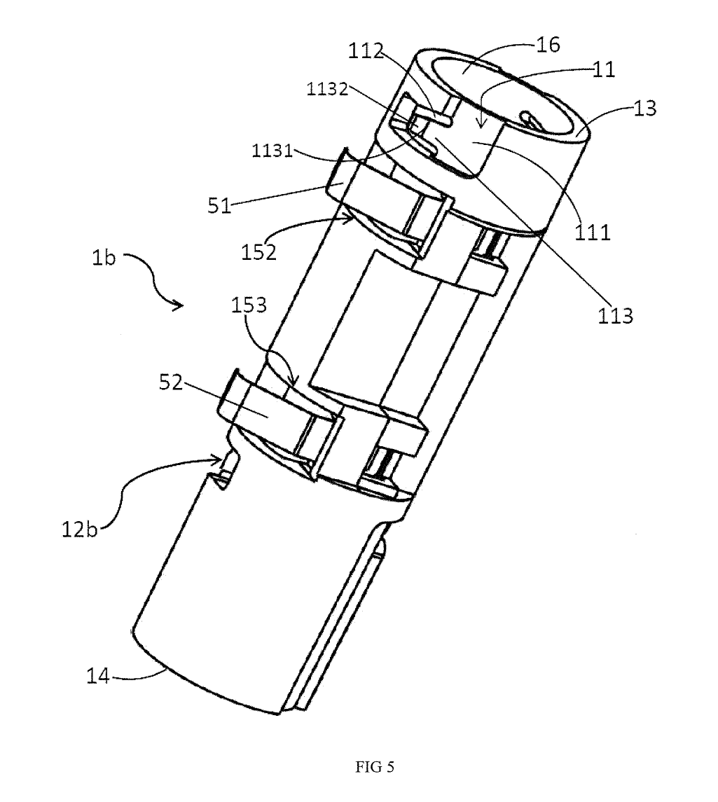

Please refer to FIG. 1 to FIG. 5, in one embodiment of the present invention, the hollow cylindrical supporting body 1 has an axial support surface 15. Moreover, the hollow cylindrical supporting body 1 has at least one upper embedded component 11 and at least one lower embedded component 12. The upper embedded component 11 and lower embedded component 12 are paralleled to the axis direction A and are opposite located in the axial support surface 15 respectively. Besides, the upper embedded component 11 is connected to a top edge portion 13 and the lower embedded component 12 is connected to a bottom edge portion 14. The upper embedded component 11 provides an upper orientation track 111 recessed from the top edge portion 13 along the axial support surface 15. The upper orientation track 111 has an upper embedding recess 112 away from the top edge portion 13 about a vertical direction. The upper orientation track 111 is further extending an upper tongue-like elastic retainer 113 in the upper embedding recess 112 via a vertical direction. The upper tongue-like elastic retainer 113 has an upper elastic curve portion 1131. Another, the upper tongue-like elastic retainer 113 has an upper elastic clip retain end 1132 along the upper elastic curve portion 1131 and away from the upper orientation track 111. The lower embedded component 12 provides a lower orientation track 121 recessed from a bottom edge portion 14 and along axial support surface 15. The lower orientation track 121 has a lower embedding recess 122 away from the bottom edge portion 14 about a vertical direction. The lower orientation track 121 is further extending a lower tongue-like elastic retainer 123 in a lower embedding recess 122 with a vertical direction. The lower tongue-like elastic retainer 123 has a lower elastic curve portion 1231. Another, the lower tongue-like elastic retainer 123 has a lower elastic retain end 1232 along the lower elastic curve portion 1231 and away from the lower orientation track 121. Certainly, in another embodiment of the present invention, but is not limited to, two upper embedded component 11, 11b and two lower embedded component 12, 12b is located in hollow cylindrical supporting body 1 with the axial support surface 15. Thus, the lower embedded component 12 and the upper embedded component 11 of the hollow cylindrical supporting body 1 are assembled precisely in the insulated outer shell 2 by the Fool-Proofing Designed of the present invention.

Furthermore, the hollow cylindrical supporting body 1 provides an inner conducting wire connection channel 171 from the top edge portion 13 to the bottom edge portion 14 along the axis direction A. The hollow cylindrical supporting body 1 provides a supporting inner wall surface 16 which is around inner conducting wire connection channel 171 along the axis direction A. The axial support surface 15 provides an upper hollow portion 181 between the upper embedded component 11 and lower embedded component 12. Another, the axial support surface 15 provides a lower hollow portion 182 along the bottom edge portion 13, and the lower hollow portion 182 is adjacent to the lower embedded component 12. The inner conducting wire connection channel 171 provides for at least one inner conducting wire passing. The upper hollow portion 181 provides one inner conducting wire passing out. In other words, one inner conducting wire, such as the second inner conducting wire IC2, could pass out the upper hollow portion 181 from the inner conducting wire channel 171. The lower hollow portion 182 provides the other inner conducting wire passing out. That is to say, the other inner conducting wire, such as the first inner conducting wire IC1, could pass out the lower hollow portion 182 from the inner conducting wire connection channel 171. Due to the inward part of the hollow cylindrical supporting body 1, the upper hollow portion 181 and lower hollow portion 182 having hollow portion, the inner conducting wire connection channel 171, the upper hollow portion 181, and the lower hollow portion 182 can be connected with each other. So, the electrical stimulation probe 100 can decrease the weight significantly by the hollow portion. Further, the inner conducting wires can be loaded inside the inner conducting wire connection channel 171 to be protected away from the high-temperature hot-melt plastic (thermosetting plastics or thermoplastics) or by the friction damage between the hollow cylindrical supporting body 1 and the insulated outer shell 2.

The plurality of the shell members of the present invention is assembled sequentially as an insulated outer shell 2 along the axis direction A to form the insulated outer shell 2 (as shown in FIG. 1 to FIG. 5). The plurality of the shell members comprises an upper shell member 21, a middle shell member 22 and a lower shell member 23. The lower shell member 23 is connected to a handheld portion 4. The lower shell member 23 has a hollow portion 24. The hollow portion 24 of the lower shell member 23 is further comprising a lower inner wall surface 231, and the lower inner wall surface 231 has at least one lower guide convex part 232 and at least one lower guide rail 233. The lower orientation track 121 guides the hollow cylindrical supporting body 1 rotationally sliding on the lower inner wall surface 231 with clockwise or counterclockwise via the lower guide part 232. The lower orientation track 121 slides and restrained on the lower guide rail 233.

That is, in the present invention, the lower guide convex part 232 could slide from the lower orientation track 121 to the lower tongue-like elastic retainer 123. The lower guide convex part 232 could slide toward the lower elastic retain end 1232 along the lower elastic curve portion 1231 and further be restrained in the lower embedding recess 122. Thus, the hollow cylindrical supporting body 1, 1b could be restrained and anchored lower inner wall surface 231 by the lower embedded component 12, 12b and the bottom edge portion 14. In other words, the Fool-Proofing Design could help matching precisely and sliding, connecting, restraining the components of the present invention by the lower guide convex part, the lower guide rail, the lower orientation track, the lower tongue-like elastic retainer, and the lower embedding recess. Furthermore, in another embodiment of the present invention, but not limit to, two lower guide convex parts 232, 232b and two lower guide rails are paired each other and sat opposite in lower inner wall surface 231. Moreover, two lower guide convex parts 232, 232b and two lower guide rails do not remain in the same vertical line in lower inner wall surface 231. In addition, two lower guide convex parts 232, 232b and two lower guide rails could guide two lower embedded components 12, 12b to slide and be restrained, locker in lower shell member 23.

The middle shell member 22 provides a middle inner wall surface 221 facing toward the hollow portion 24, and the middle inner wall surface 221 provides at least one middle guide rail 222 along axis direction A. The hollow cylindrical supporting body 1 has at least one laterally axial guide rail 151 along the axial support surface 15. The laterally axial guide rail 151 is corresponding and sliding on the middle guide rail 222. In one embodiment of the present invention, the laterally axial guide rail 151 is extended from the top edge portion 13 toward a region between the upper embedded component 11 and the lower embedded component 12 along axial support surface 15. The laterally axial guide rail 151 extended closer to the lower embedded component 12, but not reach to the bottom edge portion 14. Besides, in another embodiment of the present invention, but not limit to, paired laterally axial guide rails are corresponding and sliding on the middle guide rail 222, 222b.

In addition, the upper shell member 21 provides the upper inner wall surface 211 facing toward the hollow portion 24, and the upper inner wall surface 211 provides at least one upper guide convex part 212 along axis direction A. The upper orientation track 111 of the upper embedded component 11 is guiding and restraining the hollow cylindrical supporting body 1 rotationally moving along the upper inner wall surface 211 in a clockwise or counter-clockwise direction by the upper guide convex part 212. Thus, the upper guide convex part 212 could slide along the upper orientation track 111 to the tongue-like elastic retainer 113 and be restrained in the upper embedding recess 112 by the elasticity and press of the upper elastic curve portion 1131 and through the upper elastic clip retain end 1132. Therefore, the hollow cylindrical supporting body 1 can be restrained and locked in the upper inner wall surface 211 of the upper shell member 21 by the upper embedded component 11 and the top edge portion 13. Besides, in another embodiment of the present invention, but not limit to, two upper guide convex parts 212, 212b are paired and opposite located in the upper inner wall surface 211. Moreover, two upper guide convex parts 212, 212b is guiding the upper embedded component 11, 11b to slide and be restrained in the upper shell member 21. In summary, the hollow cylindrical supporting body 1 can be corresponded, slide, restrained and locked in the lower shell member 23, the middle shell member 22, the upper shell member 21 sequentially and precisely by the lower embedded component 12, the laterally axial guide rail 151, the upper embedded component 11.

Besides, please refer to FIG. 1 to FIG. 3, the lower shell member 23 has a third upper opening 234 which is provided in opposite direction to the handheld portion 4. A third upper opening supporting ring portion 235 is a convex-shaped portion raised along a lower inner wall surface 231 and circled around the third upper opening 234. The third upper opening supporting ring portion 235 could load a third upper O ring O3 and further form a bulge as a third conducting wire entangled part 236. The top edge of the third conducting wire entangled part 236 has a notch to form a third notch portion 2361. Furthermore, the third upper opening supporting ring portion 235 has another bulge to form a third upper opening small convex part 237. The third upper opening small convex part 237 is next to the third conducting wire entangled part 236 with a specific distance, and the specific distance is about, for example, but is not limited to the outer diameter length of the inner conducting wire. The first inner conducting wire IC1 electrically connected the outer conducting wire OC would directly coil and circle around the third conducting wire entangled part 236. Also, the first inner conducting wire IC1 would circle around the third conducting wire entangled part 236 across the third notch portion 2361 from the gap between the third conducting wire entangled part 236 and the third upper opening supporting ring portion 235. Otherwise, the first inner conducting wire IC1 would circle around the third conducting wire entangled part 236 by passing through the gap between the third conducting wire entangled part 236 and the third upper opening small convex part 237. In one embodiment of the present invention, but not limit to, the first inner conducting wire IC1 can circle around the third conducting wire entangled part 236 via passing out the lower hollow portion 182 through the inner conducting wire connection channel 171. Thus, the first inner conducting wire IC1 can be coiled and circled around the third conducting wire entangled part 236 effectively and tightly. Besides, the first inner conducting wire IC1 can be more stably circled around the third conducting wire entangled part 236 with the third notch portion 2361 and/or the third upper opening small convex part 237. Due to the third conducting wire entangled part 236 corresponded with a lower conducting ring 32, the first inner conducting wire IC1 can further electrically connected with the lower conducting ring 32. Furthermore, the inner conducting wire can be effectively restrained in the inward part of the insulated outer shell without using hot melt plastic material by the third conducting wire entangled part 236, the third notch portion 2361, and the third upper opening small convex part 237 of the present invention.

Similarly, the middle shell member 22 has a second upper opening 223 which is provided in opposite direction to the handheld portion 4. A second upper opening supporting ring portion 224 is a convex-shaped portion raised along a middle inner wall surface 221 and circled around the second upper opening 223. The second upper opening supporting ring portion 224 could load with a second upper O ring O21 and further form a bulge as a second conducting wire entangled part 225. The top edge of the second conducting wire entangled part 225 has a notch to form a second notch portion 2251. Furthermore, the second upper opening supporting ring portion 224 has another bulge to form a second upper opening small convex part 226. The second upper opening small convex part 226 is located next to the second conducting wire entangled part 225 with a specific distance. A second inner conducting wire IC2 electrically connected to the outer conducting wire OC would directly coil and circle around the second conducting wire entangled part 225. Also, the second inner conducting wire IC2 would circle around the second conducting wire entangled part 225 across the second notch portion 2251 from the gap between the second conducting wire entangled part 225 and the second upper opening supporting ring portion 224. Otherwise, the second inner conducting wire IC2 would circle around the second conducting wire entangled part 225 by passing through the gap between the second conducting wire entangled part 225 and the second upper opening small convex part 226. In one embodiment of the present invention, the second inner conducting wire IC2 could be, but not limit to, circled around the second conducting wire entangled part 225 via passing out the upper hollow portion 181 through the inner conducting wire connection channel 171. That is, the second inner conducting wire IC2 can be more stably circled around the second conducting wire entangled part 225 with the second notch portion 2251 and/or the second upper opening small convex part 226. Due to the second conducting wire entangled part 225 corresponded to an upper conducting ring 31, the second inner conducting wire IC2 can further electrically connected with the upper conducting ring 31.

The middle shell member 22 has a second lower opening 227 which is provided in opposite direction to a second upper opening 223. A second lower opening supporting ring portion 228 is a convex-shaped portion descended along the middle inner wall surface 221 and circled around the second lower opening 227. The second lower opening supporting ring portion 228 could be loaded with a second lower O ring O22. Thus, the lower conducting ring 32 could be connected with the middle shell member 22 and the lower shell member 23 more tightly by the second lower O ring O22 and the third upper O ring O3. The present invention further has the function of waterproof and moisture-proof through the second lower O ring O22 and the third upper O ring O3.

The upper shell member 21 has a first lower opening 213 which is provided for corresponding to the second upper opening 223. A first lower opening supporting ring portion 214 is a convex-shaped portion descended along upper inner wall surface 211 and circled around the first lower opening 213. The first lower opening supporting ring portion 214 could load with a first lower O ring O1. Thus, the upper conducting ring 31 could be connected with the upper shell member 21 and the middle shell member 22 more tightly by the first lower O ring O1 and the second upper O ring O21. The present invention further having the function of waterproof and moisture-proof through the first lower O ring O1 and the second upper O ring O21. That is, the present invention could prevent the water vapor, liquid penetrate into the inside of electrical stimulation probe by connecting the upper shell member 21, the first lower O ring O1, the upper conducting ring 31, the second upper O ring O21, the middle shell member 22, the second lower O ring O22, the lower conducting ring 32, the third upper O ring O3, the lower shell member 23 tightly sequentially, so as to prevent the inner conducting wires from short-circuited resulted in moisture invading. Therefore, plural shell members and plural conducting rings could be connected tightly for each other not to use large amount or even completely eliminating the need of thermosetting plastics or thermoplastics in the present invention.

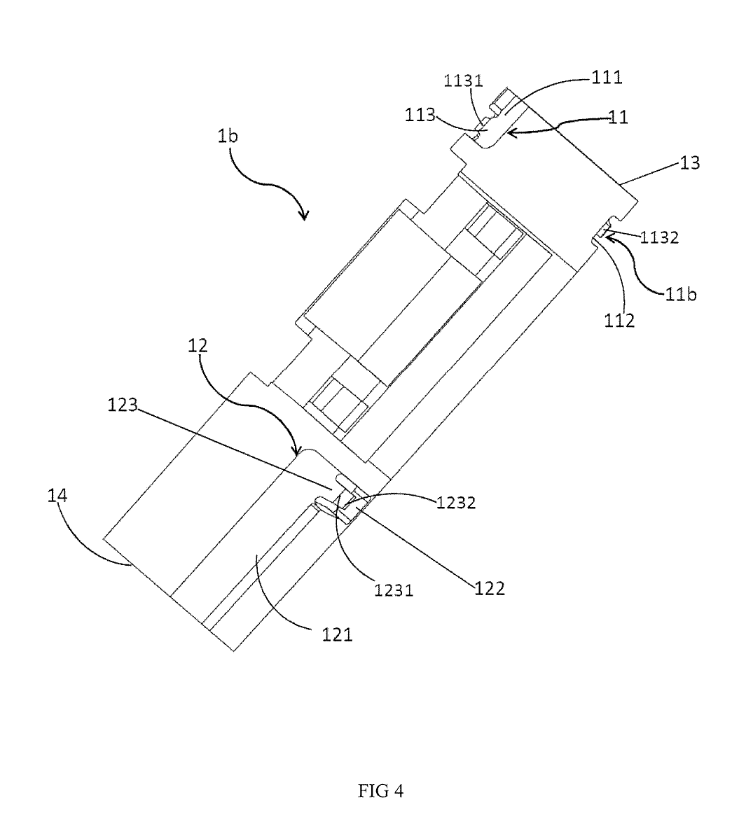

Besides, please refer to FIG. 2, FIG. 3 and FIG. 4, in another embodiment of the present invention, but not limit to, the structure between the hollow cylindrical supporting body 1b and hollow cylindrical supporting body 1 are about the same. However, the hollow cylindrical supporting body 1b further comprises an upper elastic clip recess 152 and a lower elastic clip recess 153. An upper conducting clip 51 extends from a supporting inner wall surface 16 of the hollow cylindrical supporting body 1b to the upper elastic clip recess 152, and the lower conducting clip 52 extends from the supporting inner wall surface 16 to the lower elastic clip recess 153. The plurality of inner conducting wires is electrically connected to the upper conducting clip 51 and the lower conducting clip 52. The upper conducting clip 51 and the lower conducting clip 52 is corresponded to and electrically connected with the plurality of conducting members. For example, the plurality of conducting members comprise, but not be limited to, the upper conducting ring 31 and the lower conducting ring 32.

In another embodiment of the present invention, an outer conducting wire connection channel 172 is a short concave groove along the axial support surface 15 and the axis direction A. The inner conducting wires could pass through the outer conducting wire connection channel 172 and further could circle around the third conducting wire entangled part 236 and the second conducting wire entangled part 225.

The handheld portion 4 could be screwed with the lower shell member 23. Then a small O ring O4 could provide a water-proof function for connecting with the lower shell member 23 and the handheld portion 4. Furthermore, the outer conducting wire OC connect with the handheld portion 4, and further pass through handheld portion 4 to form the inner conducting wires. One inner conducting wire, such as the first inner conducting wire IC1 could pass through the inner conducting wire connection channel 171 and circle around the third conducting wire entangled part 236 from the lower hollow portion 182. Meanwhile, the other inner conducting wire, such as the second inner conducting wire IC2 could pass through the inner conducting wire connection channel 171 and circle around the second conducting wire entangled part 225 from the upper hollow portion 181. The lower conducting ring 32 is corresponded to the third conducting wire entangled part 236 and the upper conducting ring 31 is corresponded to the second conducting wire entangled part 225 respectively. The inner conducting wires are coiled and supported by the third conducting wire entangled part 236 and the second conducting wire entangled part 225 respectively and electrically connected with the lower conducting ring 32 and the upper conducting ring 31 by outer electrical power. The insulated outer shell 2 is an electrical insulator made by electrical insulating properties and/or insulating materials. The outer electrical power could supply electricity for conducting members, such as the upper conducting ring 31 and the lower conducting ring 32 through outer conducting wire and the inner conducting wires delivered. For example, but not be limited to, the low frequency and/or heat could safely and reliably induce for physical therapy or/and electrotherapeutic modalities on the body surface or body cavity of an organism by the conducting member.

Please refer to FIG. 6 and FIG. 7, in another embodiment of the electrical stimulation probe 100c, 100e, the insulated outer shell formed by the plurality of collateral shell members are parallel to the axis direction A mirror-symmetrically. The insulated outer shell 2c, 2e assembly parallel to the axis direction A mirror-symmetrically by a first shell member 25, 25e and a second shell member 26, 26e. The conducting member would include a first conducting sheet 33, 33e and a second conducting sheet 34, 34e which is located in the first shell member 25, 25e and the second shell member 26, 26e respectively.

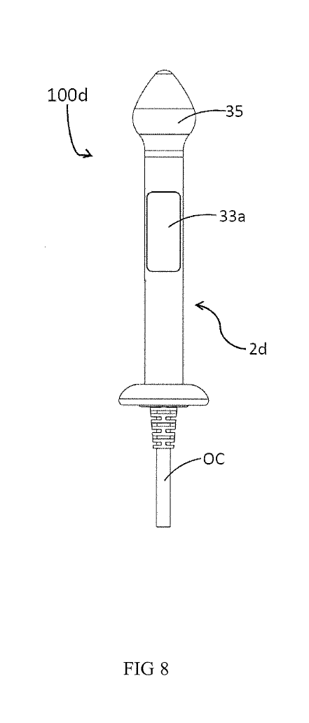

Please refer to FIG. 8, in another embodiment of the electrical stimulation probe 100d, the plurality of the shell members could be assembled to parallel the axis direction A mirror-symmetrically and assembled sequentially along the axis direction A in the same time. For example, one side of the first shell member and one side of the second shell member is cooperatively connected with a semicircular cap-shaped upper shell member. The other side of the first shell member and the other side of the second shell member could be joined together with the outer conducting wire OC. (as shown in FIG. 8). Moreover, the conducting members could include a single conducting ring 35, the first conducting sheet 33a and the second conducting sheet situated in the insulated outer shell 2d (as shown in FIG. 8). In the above-described embodiment, the plurality of the shell members which could be either assembly parallel to the axis direction A or assembled along the axis direction A matched with the hollow cylindrical supporting body precisely by the above-mentioned components of the present invention.

Summary, the plurality of conducting members and insulated outer shells are assembly parallel to the axis direction A mirror-symmetrically or non-symmetrically for the electrical stimulation probe applied to the different purposes. In another embodiment, the electrical stimulation probes are sequentially assembled by the plurality of conducting rings and insulated outer shells along axis direction A for various users.

In other word, the conducting wires were easily blown due to the injection molding process of thermosetting plastics or thermoplastics in the manufacturing process of existed probes. The large amount of thermosetting plastics or thermoplastics is needed to have the high waterproof effect for existed probes. Thus, the existing manufacture process of probes has many problems which include wasting materials, long manufacture process, low yield, etc. Additionally, the users would feel uncomfortable and inconvenient during using the existed probe. Solving the above-mentioned existed problems, the electrical stimulation probe of the present invention could assemble all the components precisely by the Fool-Proofing Designed guide rails and tracks, so the electrical stimulation probe could provide high safety, light-weighted, easily operated, and waterproof function.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. An electrical stimulation probe, comprising:
a hollowly cylindrical supporting body having at least one lower embedded component and at least one upper embedded component, wherein the lower embedded component and the upper embedded component is recessed an axial support surface in opposite to parallel an axis direction, wherein the lower embedded component and the upper embedded component is adjacent to a bottom edge portion and a top edge portion respectively;
an insulated outer shell, which is formed as a hallow portion, formed with a sliding manner to coat and support the hollow cylindrical supporting body in the hallow portion, the insulated outer shell is assembled along the axial direction or assembly parallel to the axial direction in sequence by a plurality of shell members, and the insulated outer shell is provided for installing a plurality of conductive members;
wherein the plurality of shell members are further comprising a lower shell member, the lower shell member connects to a handheld portion, the handheld portion connects to an outer conducting wire to electrically connect with an outer electrical power, wherein the lower shell member further includes a inner wall surface, the inner wall surface has at least one lower guide convex part and at least one lower guide rail, wherein the lower embedded component is guided and rotationally moved by the lower guide convex part and lower guide rail, and the lower embedded component slides and restrained on the lower guide rail, wherein the hollow cylindrical supporting body is trapped and restrained in the lower inner wall surface by the lower embedded component and the bottom edge portion.

2. The electrical stimulation probe of claim 1, wherein the lower embedded component provides a lower orientation track recessed from a bottom edge portion and along axial support surface, wherein the lower orientation track has a lower embedding recess away from the bottom edge portion about a vertical direction, wherein the lower orientation track is further extending a lower tongue-like elastic retainer in the lower embedding recess about a vertical direction.

3. The electrical stimulation probe of claim 1, wherein the upper embedded component provides an upper orientation track recessed from the top edge portion along the axial support surface, the upper orientation track has a upper embedding recess away from the top edge portion about a vertical direction, the upper orientation track is further extending an upper tongue-like elastic retainer in the upper embedding recess about a vertical direction.

4. The electrical stimulation probe of claim 3, wherein the hollow cylindrical supporting body provides a inner conducting wire connection channel from the top edge portion to the bottom edge portion along the axis direction, wherein the inner conducting wire connection channel is encompassed by a supporting inner wall surface, wherein the hollow cylindrical supporting body further comprises an upper elastic clip recess and a lower clip recess, wherein the upper conductive clip and the lower conductive clip are extended within the upper elastic clip recess and a lower elastic clip recess from the inner wall surface respectively, wherein a plurality of conducting wires electrically connect with the upper conductive clip and the lower conductive clip through the inner conducting wire connection channel, wherein the upper conductive clip and the lower conductive clip electrically connect with the plurality of conductive members respectively.

5. The electrical stimulation probe of claim 3, wherein the lower shell member has a third upper opening which is circled with a third upper opening supporting ring portion, wherein the third upper opening supporting ring portion can load a third upper O ring, wherein the third upper opening supporting ring portion further convexly form a third conducting wire entangled part, wherein the third conducting wire entangled part is corresponding to one of the plurality of conducting member, wherein the third conducting wire entangled part provides at least one inner conducting wire coiling and the inner conducting wire electrically connect with one of the plurality of conducting members, wherein the inner conducting wire electrically connect with the outer conducting wire.

6. The electrical stimulation probe of claim 5, wherein a third notch portion is recessed on the top of the third conducting wire entangled part to provide the inner conducting wire coiling.

7. The electrical stimulation probe of claim 5, further comprising a third upper opening small convex part, wherein the third upper opening small convex part is convexly located in the third upper opening supporting ring portion, wherein the third upper opening small convex part is next to the third conducting wire entangled part with a specific distance, wherein the inner conducting wire is movably passed across between the third upper opening small convex part and the third conducting wire entangled part.

8. The electrical stimulation probe of claim 5, wherein the plurality of shell members are further comprising an upper shell member, wherein an upper inner wall surface in the upper shell member is equipped with at least one upper guide convex part, wherein the upper guide convex part is movably slid and set on the upper guide rail, wherein the upper guide convex part is further locked into the upper embedding recess by passing through the tongue-like elastic retainer.

9. The electrical stimulation probe of claim 8, wherein the upper shell member has a first lower opening which is circled with a first lower opening supporting ring portion, wherein the first lower opening supporting ring portion can load a first lower O ring.

10. The electrical stimulation probe of claim 8, wherein the plurality of shell members are further comprising a middle shell member, wherein a middle inner wall surface of the middle shell members is equipped with a middle guide rail, wherein the axial support surface is set with a laterally axial guide rail, wherein the laterally axial guide rail is movably slide across the middle guide rail.

11. The electrical stimulation probe of claim 10, wherein the middle shell member has a second upper opening which is circled with a second upper opening supporting ring portion, wherein the middle shell member has a second lower opening which is circled with a second lower opening supporting ring portion, wherein the second upper opening supporting ring portion and the second lower opening supporting ring portion could be loaded with a second upper O ring and a second lower O ring respectively.

12. The electrical stimulation probe of claim 11, wherein the second upper opening supporting ring portion is further convexly set a second conducting wire entangled part, wherein the second conducting wire entangled part is corresponding to another one of the plurality of conducting member, wherein the second conducting wire entangled part is provided for the inner conducting wire coiling, and the inner conducting wire is electrically connect with another one of the plurality of conducting members.

13. The electrical stimulation probe of claim 12, further comprising a second upper opening small convex part, wherein the second upper opening small convex part is next to the second upper opening supporting ring portion with a specific distance, wherein the inner conducting wire is movably passed across between the second upper opening small convex part and the second conducting wire entangled part.

14. The electrical stimulation probe of claim 12, wherein the conducting members is selected from the group consisting of at least two conducting rings, at least two conducting sheets, and at least one conducting ring combined with at least one conducting sheet.

15. The electrical stimulation probe of claim 14, wherein one of at least two conducting rings is connected circularly with the upper shell member and the middle shell member along the axis direction, wherein the other one of at least two conducting rings is connected circularly with the middle shell member and the lower shell member along the axis direction.

16. The electrical stimulation probe of claim 14, wherein one and the other of at least two conducting sheets is parallel to the axis direction and is located relatively on the insulated outer shell respectively.

17. A hollow cylindrical supporting body for an electrical stimulation probe, wherein the electrical stimulation probe has an insulated outer shell, wherein the insulated outer shell is covering the hollow cylindrical supporting body, and the hollow cylindrical supporting body comprises:
at least one lower embedded component; and
at least one upper embedded component, wherein the lower embedded component and the upper embedded component is recessed an axial support surface in opposite to parallel to an axis direction, wherein the lower embedded component and the upper embedded component is adjacent to a bottom edge portion and a top edge portion respectively;
wherein the plurality of shell members further comprise a lower shell member and at least two conductive members, the lower shell member connects to a handheld portion, the handheld portion connects to an outer conducting wire electrically connected with an outer electrical power, wherein the lower shell member further includes a inner wall surface, the inner wall surface has at least one lower guide convex part and at least one lower guide rail, wherein the lower embedded component is guided and rotationally moved by the lower guide convex part and lower guide rail, and the lower embedded component slides and restrained on the lower guide rail, wherein the hollow cylindrical supporting body is trapped and restrained in the lower inner wall surface by the lower embedded component and the bottom edge portion.

18. The hollow cylindrical supporting body of claim 17, further comprising an inner conducting wire connection channel, an upper hollow portion, and a lower hollow portion, wherein the inner conducting wire connection channel is penetrated from the top edge portion to the bottom edge portion through the hollow cylindrical supporting body along the axis direction, wherein the axial support surface is provided the upper hollow portion between the upper embedded component and lower embedded component, wherein the axial support surface provides a lower hollow portion along the bottom edge portion, wherein the lower hollow portion is adjacent to the lower embedded component, wherein the inner conducting wire connection channel is provided for at least one inner conducting wire passing through, wherein the upper hollow portion is provided one inner conducting wire passing out from the inner conducting wire connection channel, wherein the lower hollow portion is provided the other inner conducting wire passing out from the inner conducting wire connection channel.

19. The hollow cylindrical supporting body of claim 17, wherein the lower embedded component and the upper embedded component provide a lower orientation track and an upper orientation track recessed form the bottom edge portion and the top edge portion along the axial support surface respectively, wherein the lower orientation track and the upper orientation track have a lower embedding recess and an upper embedding recess away from the bottom edge portion and the top edge portion about a vertical direction respectively, wherein the lower orientation track and the upper orientation track are further extending a lower tongue-like elastic retainer and an upper tongue-like elastic retainer in the lower embedding recess and the upper embedding recess about a vertical direction, wherein the lower tongue-like elastic retainer has a lower elastic curve portion and a lower elastic retain end, wherein the upper tongue-like elastic retainer has an upper elastic curve portion and an upper elastic retain end.

20. The hollow cylindrical supporting body of claim 17, further comprising an outer conducting wire connection channel, wherein the outer conducting wire connection channel is set on the axial support surface along the axis direction, wherein the outer conducting wire connection channel is provided for at least one inner conducting wire passing.
